(19) United States
(12) Patent Application Publication
(10) Pub. No.: US 2014/0114242 A1
(43) Pub. Date: Apr. 24, 2014

(54) INTRAJUGULAR BALLOON CATHETER

(71) Applicant: Eberhard-Karls-Universitaet Tuebingen Universitaetsklinikum, Tuebingen (DE)

(72) Inventor: Veit-Simon ECKLE, Tuebingen (DE)

(21) Appl. No.: 14/145,744

(22) Filed: Dec. 31, 2013

Related U.S. Application Data
(63) Continuation of application No. PCT/EP2012/063023, filed on Jul. 4, 2012.

(30) Foreign Application Priority Data
Apr. 5, 2011 (DE) 10 2011 106 676.8

(51) Int. Cl. A61B 17/12; A61M 25/00; A61M 25/10

(52) U.S. Cl. CPC A61B 17/12109; A61M 25/10; A61M 25/0026; A61B 17/12136; A61B 17/1204
USPC 604/96.01

(57) ABSTRACT

The present invention relates to the use of a catheter system for avoiding air embolisms in intracranial interventions and to the catheter system itself; the latter has a first and a second catheter main body, each with a longitudinal axis, a proximal end and a distal end. The catheter main bodies have several lumens and lumen openings and in each case an expandable structure for blocking the bloodstream in blood vessels.

### CROSS REFERENCES TO RELATED APPLICATIONS

This application is a continuation of international patent application PCT/EP2012/063023, filed on Jul. 4, 2012 designating the U.S., which international patent application has been published in English language and claims priority from German patent application DE 10 2011 106 676.8, filed on Jul. 5, 2011. The entire contents of these priority applications are incorporated herein by reference.

### BACKGROUND OF THE INVENTION

The present invention relates to a catheter system for insertion into blood vessels of a patient, in particular into the jugular veins, wherein the catheter system has a first catheter main body, with a longitudinal axis, a proximal end and a distal end. Moreover, the first catheter main body contains three lumens, which extend at least in part from the proximal end of the catheter main body as far as the distal end.

The three-lumen catheters described previously are used in particular in neurosurgical and other medical interventions on the open cranium. In these interventions, a sitting or half-sitting position of the patient is often required in which the operating site usually lies higher than the heart. Although this position, especially in operations performed on the posterior region of the cranium, has the advantage, among others, of permitting better access to the operating site and improved drainage of cerebrospinal fluid and reduced blood loss, the risks to the patient in such a position are many. For example, during the surgical incision of venous vessels, such a position can cause an underpressure in the vessels, and this can cause air to enter the bloodstream and the lungs and lead to life-threatening embolisms.

In patients with a rudimentary link between the left and right heart (patent foramen ovale (PFO)), there is also the danger of air passing into the right heart and thereby into arterial vessels, which can trigger a stroke. The incidence of PFO in the general population is ca. 25% (see, for example, Fathi et al., “Patent foramen ovale and neurosurgery in sitting position: a systematic review”, British Journal of Anaesthesia 102(5):588-96 (2009)).

The incidence of air embolisms in neurosurgical interventions performed on patients in a sitting/half-sitting position is at least 45% (Black et al., “Outcome Following Posterior Fossa Craniectomy in Patients in the Sitting or Horizontal Positions”, Anesthesiology 69:49-56 (1988)). Therefore, in addition to the standard monitoring customary in anaesthesiology, other measures are also recommended when performing operations with the patient in a sitting or half-sitting position, in order to ensure that air embolisms are detected reliably and with precision. To this end, in all craniotomy procedures, transesophageal echocardiography is presently used for early detection of a venous embolism, or alternatively Doppler sonography (Mirski et al., “Diagnosis and Treatment of Vascular Air Embolism”, Anaesthesiology 106:164-77 (2007)).

If an air embolism has been detected, brief manual jugular compression in the neck area is presently recommended, which is intended, on the one hand, to prevent further entry of air and, on the other hand, to make it easier for the surgeon to find the site where air has gained entry (see Mirski et al., loc. cit.).

Such manual compression of the jugular veins can lead to complications such as fractures of the hyoid bone, pressure on the carotid arteries and reduced supply of blood to the brain or entrainment of atheromatous plaque into the carotid arteries, with associated danger of a stroke. In addition, this manual compression is not especially reliable and can also have different effects from patient to patient/from user to user.

### SUMMARY OF THE INVENTION

Since alternative techniques or equipment are not described in the literature, there is a great need to make available a method or a device with which air embolisms can be efficiently avoided regardless of the particular patient who is to be treated.

According to the invention, this and other objects are achieved by a catheter system which, in addition to the three lumens in the first catheter main body with in each case a proximal, distal and medial lumen opening, also comprises, between proximal lumen opening and medial lumen opening, a first radially expandable structure for blocking a vessel, which structure is expandable via a fourth lumen that extends from the proximal end of the catheter main body as far as the expandable structure, wherein the catheter system has a second catheter main body, with a longitudinal axis, a proximal catheter main body end and a distal catheter main body end, and with a first catheter main body lumen, which extends from the proximal catheter main body end as far as a proximal catheter main body lumen opening, a second catheter main body lumen, which extends from the proximal catheter main body end as far as a distal catheter main body lumen opening that lies distally from the proximal catheter main body lumen opening, and wherein moreover a second radially expandable structure for blocking a blood vessel is provided between proximal catheter main body lumen opening and distal catheter main body lumen opening, said structure being expandable via a third catheter main body lumen.

The problem addressed by the invention is thereby completely solved.

With the catheter system according to the invention, a device is made available with which, if necessary, the lumen of the internal jugular veins can be completely closed and, in this way, the venous return to the heart from intracranial vessels can be prevented. The entry of air into the right heart and arterial vessels, and thus the triggering of a stroke, can be successfully and efficiently avoided in this way. By expansion of the expandable structures of the two catheter main bodies, it is also possible to simultaneously close the two jugular veins, analogously to the manual compression method.

Likewise, the use of the catheters provides surgical advantages for the operating surgeon since, if necessary, the lumens of the internal jugular veins can be completely closed and, in this way, it is possible to detect as yet unclosed venous vessels through the return flow of blood into the operating site.

The use of the catheter system according to the invention additionally affords the advantage that the catheter can be used at the same time to perform other catheter-assisted functions. Thus, in addition to the use according to the invention for avoiding air embolisms, it is also possible to use the catheter for intraoperative and postoperative delivery of vaso-active drugs, for example, and for delivery of liquid. There is also the possibility of using the catheter at the same time to measure jugular and mixed venous oxygen saturation.

If a venous delivery of air is discovered, the expandable structures provided in each case on the catheter main bodies make it possible, in addition to the functions already offered by a three-way catheter as such, to intervene quickly, efficiently and in a manner which is gentle on the patient and to close the vessels by expansion of the expandable structure and thereby avoid air entering the heart.

A “catheter” or “catheter main body” is understood here as any tubular or hose-like device with which access can be gained to blood vessels. The catheter can have different diameters adapted to the particular blood vessel and can be made of different materials, for example plastics, rubber, silicones, metals or glass. Such a catheter generally has a catheter tip, with which the catheter is inserted into the blood vessel, a lumen and connectors, i.e. attachment possibilities, for example Luer lock attachments, for hoses or syringes.

In addition, the terms “catheter” and “catheter main body” are used synonymously here, i.e. when the term “catheter” is used, it generally denotes a catheter main body, and vice versa, in contrast to the “catheter system” which, according to the present invention, has at least two catheters or catheter main bodies.

A “radially expandable structure” is understood here as any structure which is able, for example through delivery of a fluid or by mechanical expansion, to be converted from a non-expanded state to an expanded state in which it is widened in circumference relative to the non-expanded state; the expansion is preferably reversible, i.e. the means by which the structure was caused to expand can be removed again if necessary, as a result of which the structure can be converted back to a non-expanded state. The structure is radially expandable and thus in particular stretches out relative to its circumference. The terms “expandable” and “inflatable” are used synonymously here.

The expandable structure is located, in the non-expanded state, on the outside of the catheter main body, for example, and closely follows the longitudinal axis of the catheter main body. Expansion then takes place by means of fluid being delivered via a fourth lumen that is provided in the catheter main body and that is in fluid contact with the expandable structure. Alternatively, it is conceivable that the expandable structure is located in a folded formation in the wall of the catheter main body and can expand outwards through an opening provided in the wall of the catheter main body. Here, the fluid is likewise delivered via the fourth lumen, which is in fluid contact with the expandable structure.

Before and during regular use, the expandable structure is in the non-expanded or deflated state and follows the longitudinal axis of the catheter main body. As soon as delivery of air has been detected in the vein into which the catheter main body was introduced, a fluid for example is delivered via the fourth lumen provided in the catheter main body and causes the expandable structure to expand. The expandable structure is stretched out until its outer walls bear on the vessel walls with such a pressure as to ensure a safe and efficient vessel closure, but to avoid excessive pressure on the vessel wall and the danger of possible damage to the vessel.

For the expandable structure, it is possible to use any expandable material that has already been tested in the prior art for medical uses of this kind, a particularly suitable material in the present case being one chosen from the group comprising elastic plastics or super-elastic plastics. It will of course be appreciated, and it will be obvious to a person skilled in the art, that the material has to have a strength allowing successful closure of the vessel, while at the same time it must be ensured that the material has a tear strength in relation to the pressure exerted on the expandable structure. The material can be opaque or clear material.

The words “proximal” and “proximally” refer here to that direction or that area of the catheter system closer to the operator and further from the heart. Correspondingly, the words “distal” and “distally” refer to that area or that direction further from the operator and closer to the heart. Accordingly, the “distal end” of a catheter main body generally denotes the end with the catheter tip, and the “proximal end” denotes the end via which connections are established for the lumens of the catheter.

In the catheter system according to the invention, the first lumen opening of the first catheter main body is located proximally from the first expandable structure; this first lumen opening can be used, for example, to test the catheter system. Upon inflation/expansion of the expandable structure, the outer wall of the structure bears on the vessel wall and thereby blocks the latter. As a result, the pressure increases at the proximal lumen opening. Moreover, upon expansion of the structure, the pressure falls at the medial or second lumen opening and at the distal or third lumen opening of the catheter system located in the distal direction from the expandable structure, since the venous return of the blood is suppressed by the expanded structure. These pressure differences can be detected and measured via the lumens in fluid contact with the lumen openings and via devices connected thereto in the proximal direction.

According to an aspect of the catheter system according to the invention, the first catheter main body is inserted into the right internal jugular vein and the second catheter main body is inserted into the left internal jugular vein. Accordingly, the first catheter main body is here also referred to as the right-side catheter or catheter main body, and the second catheter main body is referred to as the left-side catheter or catheter main body. If need be, i.e. if air is detected or if it is considered surgically necessary, the two jugular veins can be closed via the expandable structures provided on each catheter main body or via the expansion/inflation of said structures.

According to another aspect of the catheter system according to the invention, the second catheter main body has a length shorter than the first catheter main body.

This embodiment has the advantage of avoiding contact between the two catheter main bodies.

The length of the catheter main bodies is preferably dictated by the anatomical length of the respective internal jugular veins into which the catheter main bodies are to be introduced. The distal end of the first catheter main body, that is to say of the right-side catheter main body (since it is to be inserted into the right internal jugular vein), can reach almost as far as the right atrium of the heart.

It is also important that the expandable structures of the two catheter main bodies each come to lie in the respective jugular vein and that the catheter main bodies are inserted into and through the jugular veins in such a way that the expandable structures are not positioned in the brachiocephalic veins, so as not to affect the outflow area of the subclavian veins. This is particularly important, since the patients' arms are generally fitted with venous access ports through which medication or liquid can be administered. If an expandable structure of the catheter were also to occlude the subclavian vein, this would have to be visible on the infusion drop counter on the arm in question: a decrease in the drop speed would in this case be detected. The expandable structure can then be deflated and the catheter pulled out slightly in the deflated state. The expandable structure can then be inflated or expanded again. The correct fit of the expandable structure can in turn be verified using the infusion drop counter on the arm, this correct fit being present when the drop speed is no longer adversely affected.

The tip, i.e. the distal end, of the first or right-side catheter preferably comes to lie like a conventional central venous catheter in the superior vena cava, in order to be able to perform the known functions of a central venous catheter (CVC).

The catheter tip, i.e. the distal end, of the second or left-side catheter can extend into the brachiocephalic vein but should be short enough to ensure that it does not interact mechanically with the right-side catheter.

In a further embodiment, the expandable structure is in each case a balloon, and if the expandable structure is in each case expandable by a fluid. The fluid is, for example, chosen from air, oxygen, distilled water and saline, in particular 0.9% strength saline solution.

The invention further relates to a method for preventing air embolisms during intracranial interventions, where a catheter system is used in a patient undergoing an intracranial intervention, which catheter system has a first catheter main body, with a longitudinal axis, a proximal end and a distal end, wherein the catheter main body contains a first lumen, which extends from the proximal end of the catheter main body as far as a proximal lumen opening, a second lumen, which extends from the proximal end of the catheter main body as far as a medial lumen opening that lies distally from the proximal lumen opening, and a third lumen, which extends from the proximal end of the catheter main body as far as a distal lumen opening that is in turn provided distally from the medial lumen opening, and wherein an expandable structure for blocking the vessel is provided between proximal lumen opening and medial lumen opening, said structure being expandable via a fourth lumen, which extends from the proximal end of the catheter main body as far as the expandable structure, for avoiding air embolisms in intracranial interventions. The catheter system provided for the use according to the invention moreover has a second catheter main body with a longitudinal axis, a proximal catheter main body end and a distal catheter main body end, and with a first catheter main body lumen, which extends from the proximal catheter main body end as far as a proximal catheter main body lumen opening, a second catheter main body lumen, which extends from the proximal catheter main body end as far as a distal catheter main body lumen opening that lies distally from the proximal catheter main body lumen opening, and wherein moreover a second radially expandable structure for blocking a blood vessel is provided between proximal catheter main body lumen opening and distal catheter main body lumen opening, said structure being expandable via a third catheter main body lumen.

By means of the above-described catheter system according to the invention, which is employed in the use according to the invention and in a corresponding method according to the invention for avoiding air embolisms in intracranial interventions and with which the jugular veins can be closed quickly and efficiently, an effective alternative to the manual compression method is made available for the first time with which air embolisms can be reliably avoided.

It will be appreciated that the aforementioned features and those still to be explained below can be used not only in the respectively cited combination but also in other combinations or singly, without departing from the scope of the present invention.

### BRIEF DESCRIPTION OF THE DRAWINGS

An illustrative embodiment of the invention is shown in the attached drawing and is described in more detail below with reference to this drawing, in which:

FIG. 1 shows a schematic view, not true to scale, of an embodiment of a catheter system according to the invention, with a first (A) and second (B) catheter main body;

FIG. 2 shows cross sections at the positions 2A, 2B and 2C indicated in FIG. 1A;

FIG. 3 shows cross sections at the positions 3A, 3B and 3C indicated in FIG. 1B; and

FIG. 4 shows a schematic view of the embodiment of the catheter system according to the invention shown in FIGS. 1A and 1B, specifically when introduced into blood vessels of a patient.

### DESCRIPTION OF PREFERRED EMBODIMENTS

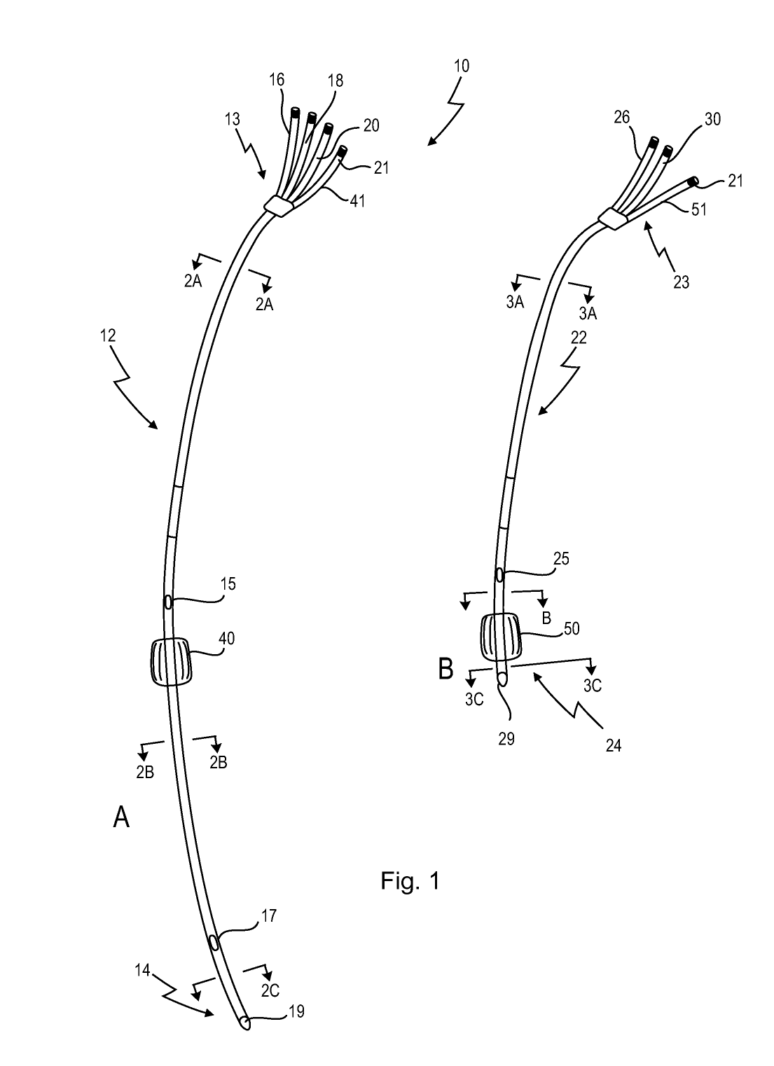

In FIGS. 1 to 4, identical features are designated by identical reference signs. In FIG. 1, reference sign 10 designates as a whole an illustrative embodiment of the catheter system according to the invention. FIG. 1A shows a first catheter main body 12, and FIG. 1B shows a second catheter main body 22. Both catheter main bodies 12 and 22 have a longitudinal axis, a proximal end 13, 23, respectively, and a distal end 14, 24, respectively. At the proximal end 13, 23, respectively, reference sign 21 schematically designates, for example in each case on one of the lumens, a connector 21 via which the lumens 16, 18, 20, 26, 30, 41 and 51, described below, of the catheter main bodies 12 and 22 can be connected in different ways. The lumens 16, 18 and 20 and 26 and 30 can be used, for example, for intravascular pressure measurement, drug delivery, liquid delivery and blood sampling.

FIG. 1A shows a proximal opening 15 of the first catheter main body 12, which proximal opening 15 is in fluid contact with a lumen 16 that extends from the proximal end 13 of the catheter main body 12 as far as the proximal lumen opening 15. Located distally from this proximal lumen opening 15, in the area of the distal end 14 of the catheter main body 12, there is a medial opening 17 and, situated further in the distal direction therefrom and provided at the distal end, a distal opening 19. The medial opening 17 is in fluid contact with a second lumen 18 that extends from the proximal end 13 of the catheter main body 12 as far as the medial lumen opening 17. The distal opening 19 is in fluid contact with a third lumen 20 that extends from the proximal end 13 of the catheter main body 12 as far as the distal lumen opening 19.

Moreover, between proximal lumen opening 15 and medial lumen opening 17, the catheter main body 12 has a first expandable structure 40, which is in fluid contact with a fourth lumen 41 and can be expanded via the latter. In the view shown in FIG. 1A, the expandable structure is in the expanded state and, in this state, is suitable for blocking the blood flow in the blood vessel in which the catheter is inserted and in which the expandable structure comes to lie.

Similarly, in FIG. 1B, the second catheter main body 22 has a proximal catheter main body lumen opening 25, which is in fluid contact with a lumen 26 that extends from the proximal catheter main body end 23 as far as the proximal catheter main body lumen opening 25. Distally from this proximal catheter main body lumen opening 25, a distal catheter main body lumen opening 29 is located at the distal end. The distal catheter main body lumen opening 29 is in fluid contact with a second catheter main body lumen 30 that extends from the proximal catheter main body end 23 as far as the distal catheter main body lumen opening 29.

Moreover, between proximal catheter main body lumen opening 25 and distal catheter main body lumen opening 29, the second catheter main body 22 has, in the distal area, a second expandable structure 50, which is in fluid contact with a third catheter main body lumen 51 and can be expanded via the latter.

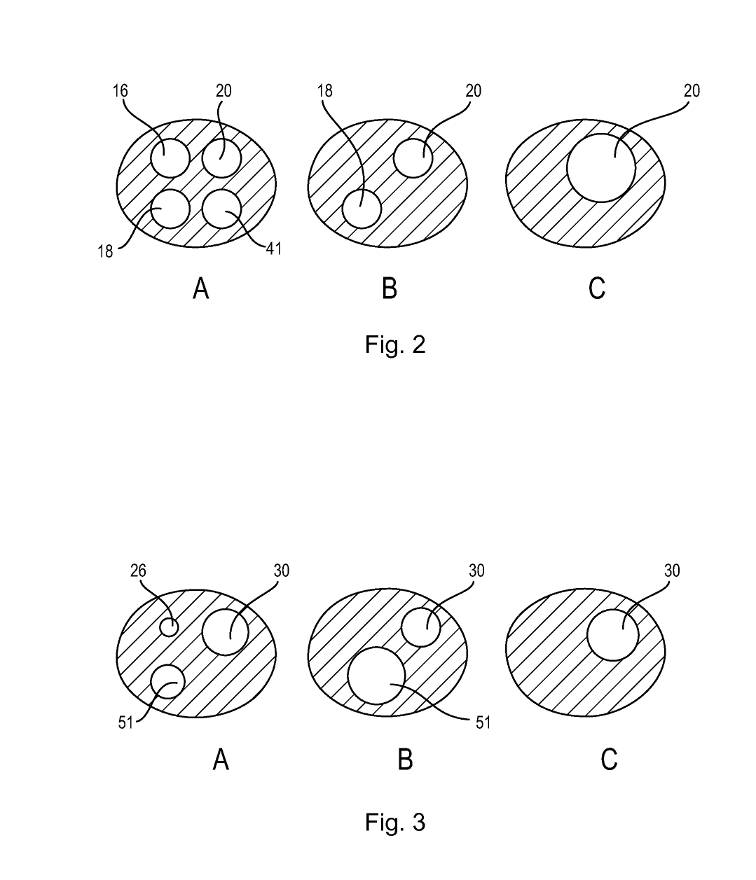

FIGS. 2 and 3 each show cross sections through the catheter main bodies 12 and 22 at the positions 2A, 2B, 2C indicated in FIG. 1A (catheter main body 12) and at the positions 3A, 3B and 3C indicated in FIG. 1B (catheter main body 22).

It will be seen from FIG. 2 that four lumens are present in the proximal area (cross section 2A) of the catheter main body 12, these four lumens being in principle of any desired arrangement and size, with FIG. 2 showing only an example. Only two lumens are still present in the area of the cross section 2B, since the first lumen 16 is in fluid contact with the first proximal lumen opening 15 and ends therein, and it is therefore no longer present in the area located further in the distal direction. Moreover, the lumen 41 likewise ends proximally from the position shown in FIG. 2B and is connected to the expandable structure 40 or ends there. Thus, in the distal area near the distal end 14, only one lumen 20 is still shown, which is in fluid contact with the lumen opening 19.

Similarly, in FIG. 3, three catheter main body lumens are present in the proximal area (cross section 3A) of the catheter main body 22, these lumens once again being of any desired arrangement and size, with the figure showing only an illustrative embodiment. Accordingly, only two catheter main body lumens 30 and 51 are still present in the area of the cross section 3B, since the third catheter main body lumen 26 ends in the catheter main body lumen opening 25, which ends proximally from the cross section 3B. Accordingly, only one catheter main body lumen 30 is still present in the area 3C, namely the one in fluid contact with the catheter main body lumen opening 29. The second expandable structure 50, which is in fluid contact with the catheter main body lumen 51, is located proximally from the position 3C.

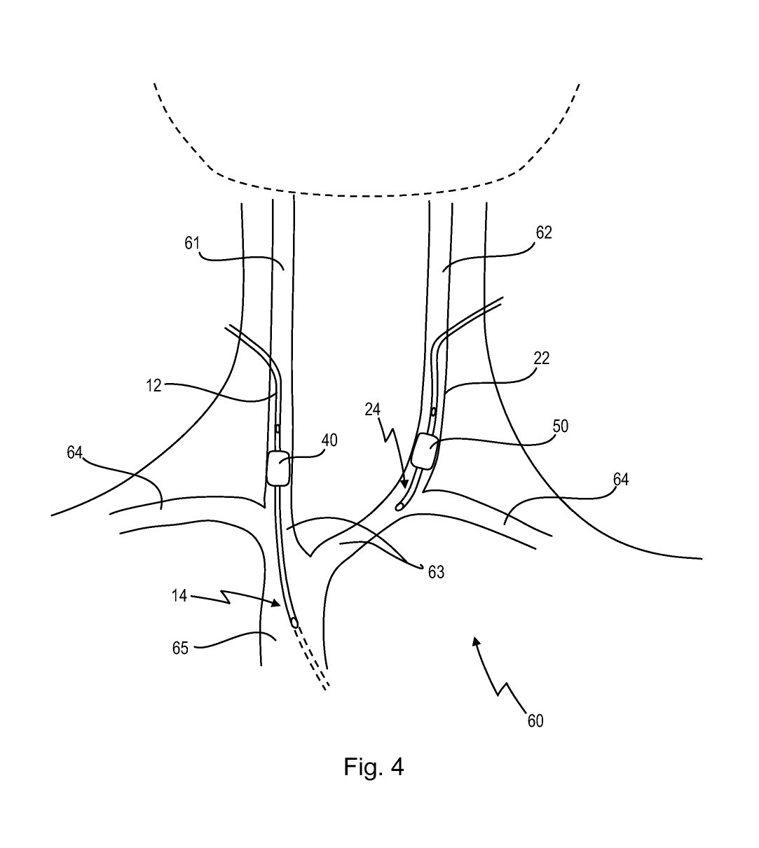

FIG. 4 shows schematically how the two catheter main bodies 12 and 22 of the catheter system 10 are introduced into a patient 60. Reference signs 61 and 62 designate the right internal jugular vein and the left internal jugular vein, respectively. The first catheter main body 12 is introduced into the right internal jugular vein 61 and is longer than the second catheter main body 22, which is introduced into the left internal jugular vein 62. The two catheter main bodies 12 and 22 do not therefore touch each other.

FIG. 4 also shows that the expandable/inflated structures 40, 50, which are in the expanded/inflated state in the figures shown, come to lie inside the jugular veins 61, 62 and, therefore, do not themselves extend, even partially, into the brachiocephalic veins 63, and, as a result, they do not completely or partially impede the venous return from the subclavian veins 64. In the embodiment shown in FIG. 4, the distal end 14 of the first catheter main body 12 ends with the catheter tip in the superior vena cava 65; the distal end 24 of the second catheter main body 22 ends with the catheter tip in the brachiocephalic vein 63.

What is claimed is:

1. A catheter system for insertion into a blood vessel of a patient, in particular into the jugular veins, wherein the catheter system has the following:
a first catheter main body, with a longitudinal axis, a proximal end and a distal end;
wherein the catheter main body contains a first lumen, which extends from the proximal end of the catheter main body as far as a proximal lumen opening,
a second lumen, which extends from the proximal end of the catheter main body as far as a medial lumen opening that lies distally from the proximal lumen opening and
a third lumen, which extends from the proximal end of the catheter main body as far as a distal lumen opening that is in turn provided distally from the medial lumen opening, and wherein
a first radially expandable structure for blocking the blood vessel is provided between proximal lumen opening and medial lumen opening, said structure being expandable via a fourth lumen, which extends from the proximal end of the catheter main body as far as the expandable structure; and wherein moreover
a second catheter main body with a longitudinal axis, a proximal catheter main body end and a distal catheter main body end is provided,
a first catheter main body lumen is provided, which extends from the proximal catheter main body end as far as a proximal catheter main body lumen opening,
a second catheter main body lumen, which extends from the proximal catheter main body end as far as a distal catheter main body lumen opening that lies distally from the proximal catheter main body lumen opening, wherein moreover
a second radially expandable structure for blocking the blood vessel is provided between proximal catheter main body lumen opening and distal catheter main body lumen opening, said structure being expandable via a third catheter main body lumen.

2. The catheter system of claim 1, wherein the second catheter main body has a shorter length than the first catheter main body.

3. The catheter system of claims 1, wherein the expandable structure is in each case a balloon.

4. The catheter system of claim 1, wherein the expandable structure is in each case expandable by a fluid.

5. The catheter system of claim 4, wherein the fluid is chosen from air, oxygen, distilled water and saline.

6. A Method for preventing air embolisms during intracranial interventions of a patient, the method comprising the steps of
applying, to a patient undergoing an intracranial intervention, a catheter system having
a first catheter main body, with a longitudinal axis, a proximal end and a distal end, wherein the first catheter main body contains a first lumen, which extends from the proximal end of the first catheter main body as far as a proximal lumen opening, a second lumen, which extends from the proximal end of the catheter main body as far as a medial lumen opening that lies distally from the proximal lumen opening, and a third lumen, which extends from the proximal end of the catheter main body as far as a distal lumen opening that is in turn provided distally from the medial lumen opening, and wherein a first radially expandable structure for blocking the vessel is provided between proximal lumen opening and medial lumen opening, said structure being expandable via a fourth lumen, which extends from the proximal end of the catheter main body as far as the expandable structure; and having further
a second catheter main body with a longitudinal axis, a proximal catheter main body end and a distal catheter main body end, wherein the second catheter main body contains a first catheter main body lumen, which extends from the proximal catheter main body end as far as a proximal catheter main body lumen opening, a second catheter main body lumen, which extends from the proximal catheter main body end as far as a distal catheter main body lumen opening that lies distally from the proximal catheter main body lumen opening, and wherein moreover a second radially expandable structure for blocking the vessel is provided between proximal catheter main body lumen opening and distal catheter main body lumen opening, said structure being expandable via a third catheter main body lumen; and
performing the intracranial intervention, thus preventing air embolisms in the patient.

7. The Method of claim 6, wherein the second catheter main body has a shorter length than the first catheter main body.
